(19) United States
(12) Patent Application Publication
(10) Pub. No.: US 2014/0214105 A1
(43) Pub. Date: Jul. 31, 2014

(54) TANDEM SERIES COUPLED IMPLANTABLE SUBCUTANEOUS CARDIOVERTER DEFIBRILLATORS AND METHOD

(71) Applicant: MEDTRONIC, INC., Minneapolis, MN (US)

(72) Inventors: Paul DeGroot, Shoreview, MN (US); Vladimir P. Nicolski, Blaine, MN (US); Theresa Whitman, Dayton, MN (US)

(73) Assignee: MEDTRONIC, INC., Minneapolis, MN (US)

(21) Appl. No.: 13/756,128

(22) Filed: Jan. 31, 2013

(51) Int. Cl. A61N 1/39

(52) U.S. Cl. CPC A61N 1/3956
USPC 607/5

(57) ABSTRACT

An implantable subcutaneous cardioverter defibrillator system for therapeutically stimulating a portion of a patient's body. First and second implantable subcutaneous cardioverter defibrillators are coupled together with a conductor. The first implantable subcutaneous cardioverter defibrillator is configured to deliver therapeutic energy in a first polarity between a first electrode and the conductor. The second implantable subcutaneous cardioverter defibrillator is configured to deliver therapeutic energy in a second polarity, opposite of said first polarity, between a second electrode and the conductor. The system is configured to synchronize delivery of the therapeutic energy by the first implantable subcutaneous cardioverter defibrillator and delivery of the therapeutic energy by the second implantable subcutaneous cardioverter defibrillator.

### FIELD

The present invention relates generally to implantable subcutaneous cardioverter defibrillators and, more particularly, to the use of multiple implantable subcutaneous cardioverter defibrillators in a single patient.

### BACKGROUND

Implantable cardioversion defibrillators, well known in the art, are small, battery-powered devices typically utilized to provide potentially life preserving therapy in patients who are at risk of sudden cardiac death due to ventricular fibrillation and/or ventricular tachycardia, among other potential cardiac ailments, e.g., other atrial and ventricular arrhythmias, bradycardia and congestive heart failure.

While some implantable cardioversion defibrillators are implanted much like conventional implantable pacemakers, some implantable cardioversion defibrillators are implanted subcutaneously with relatively minimal surgical intrusion.

U.S. Patent Application Publication No. 2009/0053180, Tandem Cardiac Pacemaker System, discloses pacemaker systems comprising an electronic pacemaker and a biological pacemaker. Thus, the tandem devices include a single electrical pacemaker and a single biological pacemaker.

An example of an implantable subcutaneous cardioverter defibrillator (“subQ ICD”) is described in U.S. Patent Publication No. 2006/0247688, Olson et al, assigned to Medtronic, Inc., Minneapolis, Minn. Such subQ ICDs provide distributed cardioversion defibrillation sense and stimulation electrodes for delivery of cardioversion defibrillation shock and pacing therapies across the heart when necessary. Embodiments disclosed include the use of dual ICDs implantable in a single patient.

Patent Cooperation Treaty Patent Application No. WO2004/047919, Subcutaneous Implantable Cardioverter Defibrillator, also discloses implantable cardioverter defibrillators that are entirely implantable subcutaneously and provide distributed cardioversion defibrillation sense and stimulation electrodes for delivery of cardioversion defibrillation shock and pacing therapies across the heart when necessary. At least two hermetically sealed housings coupled together by cable support first, second and, optionally, third cardioversion defibrillation electrodes.

Since subcutaneous cardioverter defibrillators typically utilize electrodes that are either positioned on the housing of such subcutaneous cardioverter defibrillators or nearby, due to the minimally invasive techniques used to implant the devices, the electrodes are located relatively far away from heart tissue which is the intended recipient of the devices therapy. Thus, any field developed by the electrodes from such subcutaneous cardioverter defibrillators must necessarily be larger or stronger in order to have an equal therapeutic effect on the patient. In order to have an equal therapeutic effect on the patient, the subcutaneous cardioverter defibrillators need to create a larger or stronger field. The creation of a larger or stronger field generally translates into such subcutaneous cardioverter defibrillators being either larger and bulkier or having a shortened battery life, or both. The high shock voltage and energy requirements for extravascular defibrillation may result in capacitor volumes and device size increases that impede healing of device pocket and patient comfort.

### SUMMARY

If implantable cardioverter defibrillators are implanted subcutaneously, then generally more energy is required for delivery of the therapeutic output in order to obtain the same or a similar therapeutic effect on the patient. As an example, it may be necessary to increase both the battery capacity, and hence, physical battery size, and energy storage capacity, e.g., storage capacitors, in order to deliver a stronger or larger field at an increased distance from the heart.

A typical deeply implantable cardioverter defibrillator may have an energy delivery of thirty-five joules and 750 Volts for cardioversion defibrillation. An implantable subcutaneous cardioverter defibrillator may require approximately double that amount of energy to around seventy joules and 1.5 kiloVolts for cardioversion defibrillation.

While two implantable devices may be used, as illustrated in the prior devices discussed above, just utilizing multiple devices does not provide doubled voltage. Using two standard implantable subcutaneous cardioverter defibrillators results in two devices being operated in parallel, essentially parallel capacitance which would increase the duration of current delivery but, unfortunately, does not double the voltage.

In order to double the voltage of therapy, two (or more) implantable subcutaneous cardioverter defibrillators are essentially coupled in series, i.e., the devices' high voltage output circuits are coupled in series utilizing the patient's body as part of the series circuit. This results in an effective doubling of the energy of the therapeutic capacitance coupled in series which approximately doubles the voltage of the therapeutic subcutaneous cardioversion defibrillation while keeping the devices at their same individual size and halving the capacitance.

In an embodiment, an implantable subcutaneous cardioverter defibrillator system is disclosed for therapeutically stimulating a portion of a patient's body. A conductor is operatively coupled between a first implantable subcutaneous cardioverter defibrillator and a second implantable subcutaneous cardioverter defibrillator. The first implantable subcutaneous cardioverter defibrillator is configured to deliver therapeutic energy in a first polarity between a first electrode and the conductor. The second implantable subcutaneous cardioverter defibrillator is configured to deliver therapeutic energy in a second polarity, opposite of the first polarity, between a second electrode and the conductor. The system is configured to synchronize delivery of the therapeutic energy by the first implantable subcutaneous cardioverter defibrillator and delivery of the therapeutic energy by the second implantable subcutaneous cardioverter defibrillator.

In an embodiment, the therapeutic energy from one of the first implantable subcutaneous cardioverter defibrillator and the second implantable subcutaneous cardioverter defibrillator passes through the portion of the patient's body to another of the first implantable subcutaneous cardioverter defibrillator and the second implantable subcutaneous cardioverter defibrillator.

In an embodiment, the therapeutic energy delivery by the first implantable subcutaneous cardioverter defibrillator is additive to the therapeutic energy delivered by the second implantable subcutaneous cardioverter defibrillator.

In an embodiment, the first implantable subcutaneous cardioverter defibrillator includes a first electrode on a first housing. The second implantable subcutaneous cardioverter defibrillator includes a second electrode on a second housing.

In an embodiment, the therapeutic energy of the first implantable subcutaneous cardioverter defibrillator is delivered simultaneous with the therapeutic energy from the second implantable subcutaneous cardioverter defibrillator.

In an embodiment, the portion of the patient's body comprises a majority of myocardial tissue of the patient.

In an embodiment, the therapeutic energy from the first subcutaneous cardioverter defibrillator and the therapeutic energy from the second subcutaneous cardioverter defibrillator is applied to the majority of myocardial tissue of the patient in a single vector.

In an embodiment, the therapeutic energy delivered to the majority of the myocardial tissue of the patient is approximately equal to the therapeutic energy available from the first subcutaneous cardioverter defibrillator added to the therapeutic energy available from the second subcutaneous defibrillator.

In an embodiment, the therapeutic energy delivered to the majority of the myocardial tissue of the patient is approximately double the therapeutic energy available from one of the first subcutaneous cardioverter defibrillator and the second subcutaneous cardioverter defibrillator.

In an embodiment, each of the first implantable subcutaneous cardioverter defibrillator and the second implantable subcutaneous cardioverter defibrillator has an internal energy source. A therapeutic energy delivery module operatively is coupled to the internal energy source. Control circuitry is operatively coupled to the therapeutic energy delivery module and configured to control delivery of the therapeutic energy.

In an embodiment, each of the first implantable subcutaneous cardioverter defibrillator and the second implantable subcutaneous cardioverter defibrillator further have circuitry for a need for delivery of the therapeutic energy.

In an embodiment, the conductor is electrically isolated from tissue of the body of the patient.

### FIGURES

FIG. 1 illustrates the subcutaneous implantation of two implantable subcutaneous cardioverter defibrillators in accordance with an embodiment;

FIG. 2 is a schematic block diagram illustrating the use of and connection of two implantable subcutaneous cardioverter defibrillators in accordance with an embodiment;

FIG. 3 is a more detailed block diagram illustrating the operation and connection of two implantable subcutaneous cardioverter defibrillators in accordance with an embodiment; and

FIG. 4 is a flow chart of an embodiment.

### DESCRIPTION

Implantable subcutaneous cardioverter defibrillators are well known in the art. U.S. Pat. No. 7,684,864, Olson et al, Subcutaneous Cardioverter Defibrillator, assigned to Medtronic, Inc., Minneapolis, Minn., is hereby incorporated herein by reference in its entirety.

In order to obtain an increased voltage in therapeutic energy delivered by subcutaneous cardioverter defibrillators, the high voltage output circuits of such subcutaneous cardioverter defibrillators are coupled in series to approximately double the voltage available for therapeutic energy.

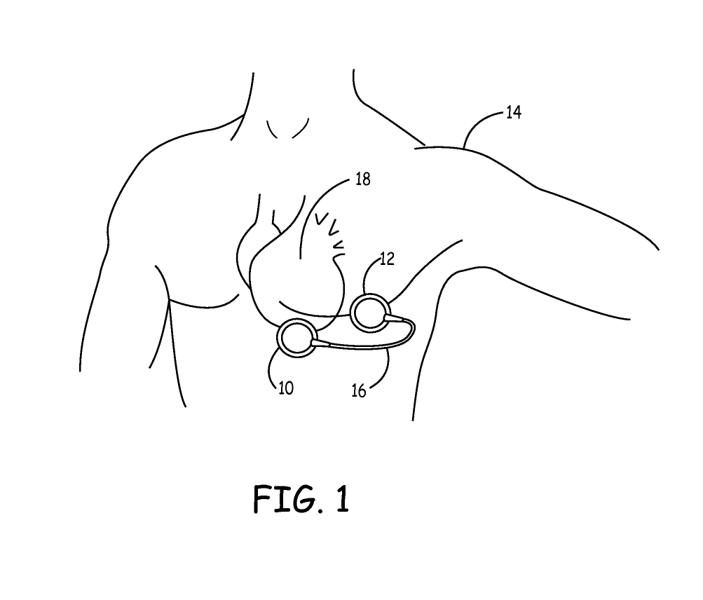

FIG. 1 illustrates implantable subcutaneous cardioverter defibrillator 10 and implantable subcutaneous cardioverter defibrillator 12 implanted subcutaneously in patient 14. Implantable subcutaneous cardioverter defibrillator 10 is coupled, either by a hardwire conductor 16 or wirelessly, to implantable subcutaneous cardioverter defibrillator 12. An electromagnetic field is created by both implantable subcutaneous cardioverter defibrillator 10 and implantable subcutaneous cardioverter defibrillator 12 with a vector of such electromagnetic field encompassing at least part of, and preferably a majority of, the heart or cardiac tissue 18. If conductor 16 is utilized, conductor 16 may be electrically isolated from the patient's body 14 preventing conductor 16 itself from being an electrode.

After implantable subcutaneous cardioverter defibrillator 10 and implantable subcutaneous cardioverter defibrillator 12 are charged, i.e., the high voltage delivery circuits are primed for delivery of therapeutic energy, both devices deliver the stored energy in synchronization, preferably simultaneously.

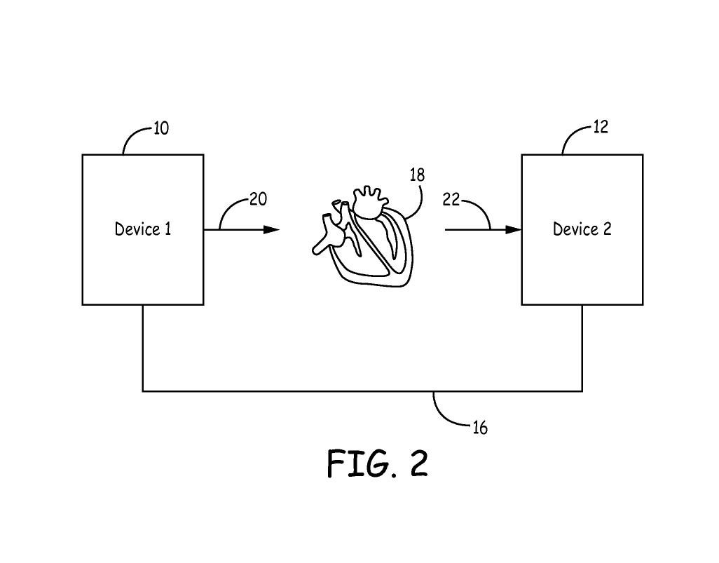

FIG. 2 is a block diagram schematic of implantable subcutaneous cardioverter defibrillator 10, implantable subcutaneous cardioverter defibrillator 12, optionally coupled with conductor 16. Implantable subcutaneous cardioverter defibrillator 10 has a single electrode 20 while implantable subcutaneous cardioverter defibrillator 12 also has a single electrode 22.

Implantable subcutaneous cardioverter defibrillator 10 is configured to deliver therapeutic energy through electrode 20 in a first polarity with respect to a reference. Implantable subcutaneous cardioverter defibrillator 12 is configured to deliver therapeutic energy through electrode 22 in a second polarity, the opposite polarity of implantable subcutaneous cardioverter defibrillator 10, with respect to the same reference. The reference in this configuration is conductor 16 which couples implantable subcutaneous cardioverter defibrillator 10 and implantable subcutaneous cardioverter defibrillator 12 together.

It should be recognized and understood that opposite polarity with respect to the energy generated by implantable subcutaneous cardioverter defibrillator 10 and the energy generated by implantable subcutaneous cardioverter defibrillator 12 refers to the polarity at a point or instance in time. For example, if the energy delivered by implantable subcutaneous cardioverter defibrillator 10 and implantable subcutaneous cardioverter defibrillator 12 results in a field that is only mono-phasic, i.e., has only one direction, then the energy generated by implantable subcutaneous cardioverter defibrillator 10 and implantable subcutaneous cardioverter defibrillator 12 would be static and opposite in polarity. However, the energy generated by implantable subcutaneous cardioverter defibrillator 10 and implantable subcutaneous cardioverter defibrillator 12 may be multi-phasic, e.g., bi-phasic or having a waveform of dual polarity, then implantable subcutaneous cardioverter defibrillator 10 and implantable subcutaneous cardioverter defibrillator 12 would be synchronized such that the energy waveform produced by each would be opposite in polarity to the other at a given point in time, preferably at any point in time. In such an embodiment, there would be coordinated, controlled or synchronized polarity switching between implantable subcutaneous cardioverter defibrillator 10 and implantable subcutaneous cardioverter defibrillator 12.

As implantable subcutaneous cardioverter defibrillator 10 is configured to deliver energy in one polarity and implantable subcutaneous cardioverter defibrillator 12 is configured to deliver therapy in the opposite polarity, with respect to conductor 16, in effect one of electrode 20 and 22 delivers energy and the other of electrodes 20 and 22 receives energy. Current exiting from implantable subcutaneous cardioverter defibrillator 10 via electrode 20, for example, passes through the patient's body 18, enters implantable subcutaneous cardioverter defibrillator 12 via electrode 22 and passes through conductor 16 completing the discharge circuit. As this effectively places implantable subcutaneous cardioverter defibrillator 10 and implantable subcutaneous cardioverter defibrillator 12 in series, the resultant energy field passing through the patient's body is approximately the additive voltage of each of implantable subcutaneous cardioverter defibrillator 10 and implantable subcutaneous cardioverter defibrillator 12 individually. As an example, implantable subcutaneous cardioverter defibrillator 10 and implantable subcutaneous cardioverter defibrillator 12 have approximately the same or similar output voltages, the voltage applied across the patient's body is approximately double the voltage of each one of implantable subcutaneous cardioverter defibrillator 10 and implantable subcutaneous cardioverter defibrillator 12 individually.

While implantable subcutaneous cardioverter defibrillator 10 and implantable subcutaneous cardioverter defibrillator 12 are illustrated in FIG. 2 as delivering therapeutic energy to a portion of or principally across cardiac tissue, it is to be recognized and understood that implantable subcutaneous cardioverter defibrillator 10 and implantable subcutaneous cardioverter defibrillator 12 could be configured to stimulate another area or other areas of the body. For example, implantable subcutaneous cardioverter defibrillator 10, or an electrode associated with implantable subcutaneous cardioverter defibrillator 10, could be positioned toward one side of the chest cavity and implantable subcutaneous cardioverter defibrillator 12, or an electrode associated with implantable subcutaneous cardioverter defibrillator 12, could be positioned toward another side of the chest cavity providing the opportunity for transvenous defibrillation.

It is also to be recognized and understood that implantable subcutaneous cardioverter defibrillator 10 and implantable subcutaneous cardioverter defibrillator 12, or electrodes associated with either of them, could be configured to provide not just a single stimulation vector. For example, one of implantable subcutaneous cardioverter defibrillator 10 or implantable subcutaneous cardioverter defibrillator 12, or both of them, could have another electrode, e.g., a subcutaneous patch or a transvenous electrode, and the additive therapeutic energy from both implantable subcutaneous cardioverter defibrillator 10 and implantable subcutaneous cardioverter defibrillator 12 could be delivered across more than one pathway, i.e., providing more than one vector.

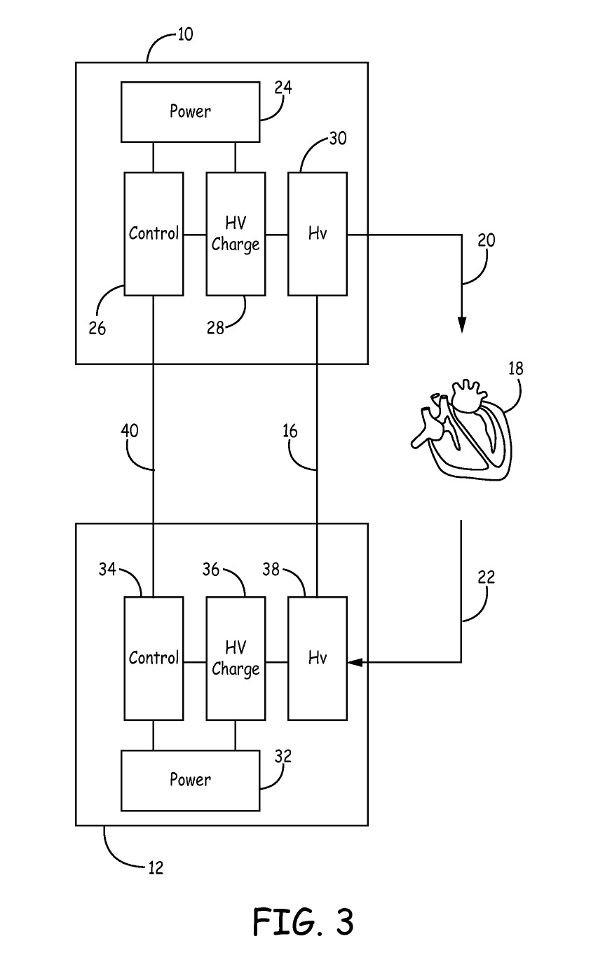

Operation of implantable subcutaneous cardioverter defibrillator 10 and implantable subcutaneous cardioverter defibrillator 12 can be seen more readily in the more detailed block diagram schematic illustrated in FIG. 3.

Implantable subcutaneous cardioverter defibrillator 10 is powered by energy source 24, typically a battery. Energy source 24 powers both control module 26 and high voltage charge circuit 28. Control module 26 conventionally controls the operation of implantable subcutaneous cardioverter defibrillator 10 and may comprise, for example, sensing circuitry to determine when to deliver therapeutic energy. Upon command of control module 26, charge circuit 28 will charge using power from energy source 24. When appropriately charged, high voltage circuit 30, typically a capacitor or a bank of capacitors, will be available to delivery energy to electrode 20.

Similarly, implantable subcutaneous cardioverter defibrillator 12 is powered by energy source 32, typically a battery. Energy source 32 powers both control module 34 and high voltage charge circuit 36. Control module 34 conventionally controls the operation of implantable subcutaneous cardioverter defibrillator 12 and may comprise, for example, sensing circuitry to determine when to deliver therapeutic energy. Upon command of control module 34, charge circuit 36 will charge using power from energy source 32. When appropriately charged, high voltage circuit 38, typically a capacitor or a bank of capacitors, will be available to delivery energy to electrode 22.

As noted with respect to FIG. 2, current exiting from implantable subcutaneous cardioverter defibrillator 10 via electrode 20, for example, passes through the patient's body 18, enters implantable subcutaneous cardioverter defibrillator 12 via electrode 22 and passes through conductor 16 completing the discharge circuit. As this effectively places implantable subcutaneous cardioverter defibrillator 10 and implantable subcutaneous cardioverter defibrillator 12 in series, the resultant energy field passing through the patient's body is approximately the additive voltage of each of implantable subcutaneous cardioverter defibrillator 10 and implantable subcutaneous cardioverter defibrillator 12 individually. As an example, implantable subcutaneous cardioverter defibrillator 10 and implantable subcutaneous cardioverter defibrillator 12 have approximately the same or similar output voltages, the voltage applied across the patient's body is approximately double the voltage of each one of implantable subcutaneous cardioverter defibrillator 10 and implantable subcutaneous cardioverter defibrillator 12 individually.

In conventional Medtronic implantable cardioverter defibrillator terminology used with Entrust™ DR/VR (Model D154ATG/D154VRC), implantable subcutaneous cardioverter defibrillator 10 may be programmed as device number 1 as AX>B, implantable subcutaneous cardioverter defibrillator 12 may be programmed as device number 2 as B>AX, with a cross connection via RV DF1 ports (“B”) of both devices using isolated cable wire, conductor 16, and a simultaneous initiation of therapy delivery.

Synchronization of implantable subcutaneous cardioverter defibrillator 10 and implantable subcutaneous cardioverter defibrillator 12 may be done using control wire 40 coupling control module 26 of implantable subcutaneous cardioverter defibrillator 10 with control module 34 of implantable subcutaneous cardioverter defibrillator 12. Alternatively, implantable subcutaneous cardioverter defibrillator 10 and implantable subcutaneous cardioverter defibrillator 12 may be synchronized wirelessly. Synchronization can also be achieved in a master-slave relationship in which one of implantable subcutaneous cardioverter defibrillator 10 or implantable subcutaneous cardioverter defibrillator 12, the master, determines if and when to deliver therapy energy and the other one of implantable subcutaneous cardioverter defibrillator 10 and implantable subcutaneous cardioverter defibrillator 12, the slave, acts on command of the master. Synchronization may also be achieved by a dual smart arrangement in which both implantable subcutaneous cardioverter defibrillator 10 and implantable subcutaneous cardioverter defibrillator 12 have the same or similar sensing circuitry and/or programming so that each of implantable subcutaneous cardioverter defibrillator 10 and implantable subcutaneous cardioverter defibrillator 12 sense the same or similar environment and make the same or similar decision as to when to deliver therapy resulting in synchronization. Also, synchronization may be achieved with one of implantable subcutaneous cardioverter defibrillator 10 and implantable subcutaneous cardioverter defibrillator 12 sensing and determining to deliver conventionally while the other of implantable subcutaneous cardioverter defibrillator 10 and implantable subcutaneous cardioverter defibrillator 12 is actuated by the energy pulse delivered by the other. In this instance, although therapy delivery will not be exactly simultaneous, therapy delivery is still synchronized. It is to be recognized and understood that the described examples of synchronization are merely exemplary and other forms of synchronization are contemplated.

It is also to be recognized and understood that synchronization does not necessarily mean simultaneity. It is not necessary that each device deliver therapy at the exact same time. Generally, synchronization occurs if therapy delivery occurs within approximately 10 milliseconds. Still preferably, synchronization can occur in less than 1 milliseconds.

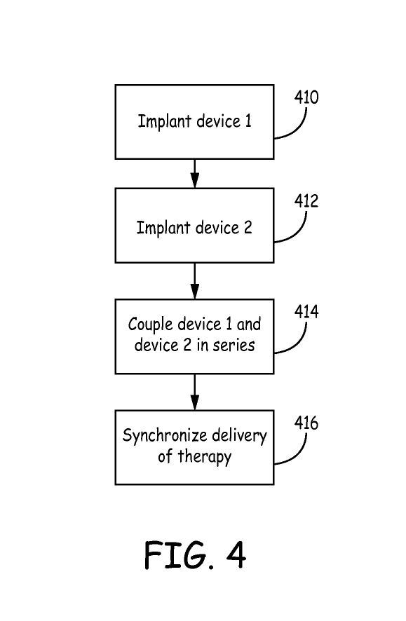

FIG. 4 is a flow diagram illustrating an embodiment. A first implantable subcutaneous cardioverter defibrillator is subcutaneously implanted 410 and configured to deliver therapeutic energy in a first polarity between an electrode and a conductor. A second implantable subcutaneous cardioverter defibrillator is subcutaneously implanted 412 and configured to deliver therapeutic energy in a second polarity between an electrode and the conductor, the second polarity being opposite to the first polarity. The high voltage circuits of the first and second implantable subcutaneous cardioverter defibrillators are coupled together 414 with the conductor acting as the second electrode on each device. Each device is configured to deliver therapy 416 on a synchronized basis. The therapeutic energy from one of the devices and the other of the devices passes through said portion of the patient's body to another device completing the discharge circuit.

Thus, embodiments of the invention are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. An implantable subcutaneous cardioverter defibrillator system for therapeutically stimulating a portion of a patient's body, comprising:
a first implantable subcutaneous cardioverter defibrillator; and
a second implantable subcutaneous cardioverter defibrillator;
said first implantable subcutaneous cardioverter defibrillator being configured to deliver therapeutic energy in a first polarity between a first electrode and a reference;
said second implantable subcutaneous cardioverter defibrillator being configured to deliver therapeutic energy in a second polarity, opposite of said first polarity, between a second electrode and said reference;
said system being configured to synchronize delivery of said therapeutic energy by said first implantable subcutaneous cardioverter defibrillator and delivery of said therapeutic energy by said second implantable subcutaneous cardioverter defibrillator.

2. The system of claim 1 further comprising a conductor operatively coupled between said first implantable subcutaneous cardioverter defibrillator and said second implantable subcutaneous cardioverter defibrillator and wherein said conductor serves as said reference.

3. The system of claim 2 wherein said system is configured to synchronize delivery of said therapeutic energy by said first implantable subcutaneous cardioverter defibrillator and delivery of said therapeutic energy by said second implantable subcutaneous cardioverter defibrillator utilizing said conductor.

4. The system of claim 1 wherein said first implantable subcutaneous cardioverter defibrillator and said second implantable subcutaneous cardioverter defibrillator deliver said therapeutic energy across a single vector.

5. The system of claim 1 wherein said first implantable subcutaneous cardioverter defibrillator and said second implantable subcutaneous cardioverter defibrillator deliver said therapeutic energy across a plurality of vectors.

6. The system of claim 1 wherein said system is configured to wirelessly synchronize delivery of said therapeutic energy by said first implantable subcutaneous cardioverter defibrillator and delivery of said therapeutic energy by said second implantable subcutaneous cardioverter defibrillator.

7. The system of claim 1 wherein said first implantable subcutaneous cardioverter defibrillator operates as a master to synchronize delivery of said therapeutic energy by said second implantable subcutaneous cardioverter defibrillator operating as a slave.

8. The system of claim 1 wherein said system is configured to deliver said therapeutic energy from one of said first implantable subcutaneous cardioverter defibrillator and said second implantable subcutaneous cardioverter defibrillator to pass through said portion of said patient's body to another of said first implantable subcutaneous cardioverter defibrillator and said second implantable subcutaneous cardioverter defibrillator

9. The system of claim 1 wherein said therapeutic energy delivery by said first implantable subcutaneous cardioverter defibrillator is additive to said therapeutic energy delivered by said second implantable subcutaneous cardioverter defibrillator.

10. The system of claim 9:
wherein said first implantable subcutaneous cardioverter defibrillator has a first housing;
wherein said second implantable subcutaneous cardioverter defibrillator has a second housing;
wherein said first housing of said first implantable subcutaneous cardioverter defibrillator includes said first electrode; and
wherein said second housing of said second implantable subcutaneous cardioverter defibrillator includes said second electrode.

11. The system of claim 9 wherein said system is configured to deliver said therapeutic energy of said first implantable subcutaneous cardioverter defibrillator simultaneous with said therapeutic energy delivered by said second implantable subcutaneous cardioverter defibrillator.

12. The system of claim 5 wherein said portion of said patient's body comprises a majority of myocardial tissue of said patient.

13. The system of claim 12 wherein said system is configured with said therapeutic energy from said first subcutaneous cardioverter defibrillator and said therapeutic energy from said second subcutaneous cardioverter defibrillator being applied to said majority of myocardial tissue of said patient in a single vector.

14. The system of claim 1 wherein said therapeutic energy delivered to said majority of said myocardial tissue of said patient is approximately equal to said therapeutic energy available from said first subcutaneous cardioverter defibrillator added to said therapeutic energy available from said second subcutaneous defibrillator.

15. The system of claim 14 wherein said therapeutic energy delivered to said majority of said myocardial tissue of said patient is approximately double said therapeutic energy available from one of said first subcutaneous cardioverter defibrillator and said second subcutaneous defibrillator.

16. The system of claim 1 wherein each of said first implantable subcutaneous cardioverter defibrillator and said second implantable subcutaneous cardioverter defibrillator comprises:
an internal energy source;
a therapeutic energy delivery module operatively coupled to said internal energy source; and
control circuitry operatively coupled to said therapeutic energy delivery module configured to control delivery of said therapeutic energy.

17. The system of claim 16 wherein each of said first implantable subcutaneous cardioverter defibrillator and said second implantable subcutaneous cardioverter defibrillator further comprises circuitry for a need for delivery of said therapeutic energy.

18. The system of claim 2 wherein said conductor is electrically isolated from tissue of said body of said patient.

19. A method of delivering therapeutic energy to a portion of a patient's body utilizing a first implantable subcutaneous cardioverter defibrillator, a second implantable subcutaneous cardioverter defibrillator, comprising the steps of:
subcutaneously implanting said first implantable subcutaneous cardioverter defibrillator configured to deliver therapeutic energy in a first polarity between a first electrode and a reference; and
subcutaneously implanting a second implantable subcutaneous cardioverter defibrillator configured to deliver therapeutic energy in a second polarity, opposite of said first polarity, between a second electrode and said reference; and
operatively coupling said first implantable subcutaneous cardioverter defibrillator and said second implantable subcutaneous cardioverter defibrillator with said reference;
synchronizing delivery of said therapeutic energy by said first implantable subcutaneous cardioverter defibrillator and with delivery of said therapeutic energy by said second implantable subcutaneous cardioverter defibrillator;
wherein said therapeutic energy from one of said first implantable subcutaneous cardioverter defibrillator and said second implantable subcutaneous cardioverter defibrillator passes through said portion of said patient's body to another of said first implantable subcutaneous cardioverter defibrillator and said second implantable subcutaneous cardioverter defibrillator.

20. The method of claim 19 wherein said operatively coupling step comprises operatively coupling said first implantable subcutaneous cardioverter defibrillator and said second implantable subcutaneous cardioverter defibrillator with a conductor coupled between said first implantable subcutaneous cardioverter defibrillator and said second implantable subcutaneous cardioverter defibrillator.

21. The method of claim 20 wherein said synchronization step is accomplished utilizing said conductor.

22. The method of claim 19 wherein therapeutic energy is delivered across a single vector.

23. The method of claim 19 wherein said therapeutic energy is delivered across a plurality of vectors.

24. The method of claim 19 wherein said synchronization step is accomplished wirelessly.

25. The method of claim 19 wherein said synchronization step is accomplished by master-slave operation between said first implantable subcutaneous cardioverter defibrillator and said second implantable subcutaneous cardioverter defibrillator.

26. The method of claim 19 wherein said therapeutic energy from one of said first implantable subcutaneous cardioverter defibrillator and said second implantable subcutaneous cardioverter defibrillator pass through said portion of said patient's body to another of said first implantable subcutaneous cardioverter defibrillator and said second implantable subcutaneous cardioverter defibrillator

27. The method of claim 19 wherein said synchronizing delivery step is accomplished with said therapeutic energy delivery by said first implantable subcutaneous cardioverter defibrillator being additive to said therapeutic energy delivered by said second implantable subcutaneous cardioverter defibrillator.

28. The method of claim 27 wherein said therapeutic energy by said first implantable subcutaneous cardioverter defibrillator is delivered from an electrode on a first housing of said first implantable subcutaneous cardioverter defibrillator and wherein said therapeutic energy by said second implantable subcutaneous cardioverter defibrillator is delivered from an electrode on a second housing of said second implantable subcutaneous cardioverter defibrillator.

29. The method of claim 28 wherein said synchronized delivery step is accomplished simultaneously with both said first implantable subcutaneous cardioverter defibrillator and said second implantable subcutaneous cardioverter defibrillator.

30. The method of claim 29 wherein said portion of said patient's body comprises a majority of myocardial tissue of said patient.

31. The method of claim 30 wherein said therapeutic energy from said first subcutaneous cardioverter defibrillator and said therapeutic energy from said second subcutaneous cardioverter defibrillator are applied to said majority of myocardial tissue of said patient in a single vector.

32. The method of claim 31 wherein said therapeutic energy delivered to said majority of said myocardial tissue of said patient is approximately equal to said therapeutic energy available from said first subcutaneous cardioverter defibrillator added to said therapeutic energy available from said second subcutaneous defibrillator.

33. The method of claim 31 wherein said therapeutic energy delivered to said majority of said myocardial tissue of said patient is approximately double said therapeutic energy available from one of said first subcutaneous cardioverter defibrillator and said second subcutaneous defibrillator.

34. The method of claim 20 wherein said conductor is electrically isolated from tissue of said body of said patient.
